(19) United States
(12) Patent Application Publication
(10) Pub. No.: US 2023/0070923 A1
(43) Pub. Date: Mar. 9, 2023

(54) Aesthetic-Enhancing Formulations And Methods Thereof

(71) Applicants: Logan D. GULLA, Costa Mesa, CA (US); Jessica N. MORRIS, Costa Mesa, CA (US); Kurt K. BELCHER, Costa Mesa, CA (US)

(72) Inventors: Logan D. Gulla, Costa Mesa, CA (US); Jessica N. Morris, Costa Mesa, CA (US); Kurt K. Belcher, Costa Mesa, CA (US)

(21) Appl. No.: 17/797,083

(22) PCT Filed: Feb. 4, 2021
(86) PCT No.: PCT/US2021/016664

§ 371 Date: Aug. 2, 2022

Related U.S. Application Data
(60) Provisional application No. 62/970,704, filed on Feb. 5, 2020.

(51) Int. Cl. A61Q 1/02; A61K 8/65

(52) U.S. Cl. CPC A61Q 1/025; A61K 8/65; A61K 2800/43

(57) ABSTRACT

Disclosed herein are aesthetic-enhancing formulations and methods thereof. For example, an aesthetic-enhancing formulation includes, in some embodiments, one or more pigments, one or more active ingredients, and one or more excipients. The one-or-more excipients includes at least a vehicle configured to evenly distribute components throughout the aesthetic-enhancing formulation, the components including the one-or-more pigments, the one-or-more active ingredients, and any other excipients other than the vehicle. In another example, a method for the aesthetic-enhancing formulation includes, in some embodiments, inserting the aesthetic-enhancing formulation into a dermal layer of skin by percutaneous punctures.

### BACKGROUND

This application claims the benefit of priority to U.S. Provisional Pat. Application No. 62/970,704, filed Feb. 5, 2020, which is incorporated by reference in its entirety into this application.

### BACKGROUND

Micropigmentation is increasingly being used for aesthetic enhancement of the body, particularly to conceal scars, stretch marks, or vitiligo patches, create or restore nipples or areolas, create or enhance hair follicles on scalps, eyebrow regions, or alopecia spots, or the like. While micropigmentation is effective for the foregoing types of aesthetic enhancement, micropigmentation consists of inserting pigments into the skin with a microneedler without any therapeutic treatment of the underlying conditions leading people to seek the foregoing types of aesthetic enhancement in the first place. Aesthetic enhancement including therapy is needed to treat such underlying conditions more effectively.

Disclosed herein are several embodiments of aesthetic-enhancing formulations or methods that meet at least the foregoing need.

### SUMMARY

Disclosed herein is an aesthetic-enhancing formulation configured for insertion into a dermal layer of skin by percutaneous punctures. The aesthetic-enhancing formulation includes, in some embodiments, one or more pigments, one or more active ingredients, and one or more excipients. The one-or-more excipients includes at least a vehicle configured to evenly distribute components throughout the aesthetic-enhancing formulation, the components including the one-or-more pigments, the one-or-more active ingredients, and any other excipients other than the vehicle.

In some embodiments, each pigment of the one-or-more pigments is a plant-based pigment.

In some embodiments, the one-or-more pigments are chosen to match a natural color of human hair.

In some embodiments, the one-or-more pigments are chosen to match a natural color of human skin.

In some embodiments, the one-or-more active ingredients include a local anesthetic.

In some embodiments, the one-or-more active ingredients include stem cells, platelet-rich plasma, extracellular vesicles, a peptide hormone, a growth factor, collagen, nutrients, or a combination thereof.

In some embodiments, the vehicle is water, hamamelis water, ethanol, isopropyl alcohol, propylene glycol, glycerol, or a combination thereof.

In some embodiments, the aesthetic-enhancing formulation further includes one or more stabilizers configured to prevent degradation of any component or combination of the components of the aesthetic-enhancing formulation.

In some embodiments, the one-or-more stabilizers include an antioxidant to prevent oxidative degradation of the aesthetic-enhancing formulation.

In some embodiments, the one-or-more stabilizers include an emulsifier to prevent separation of the components of the aesthetic-enhancing formulation into layers of the aesthetic-enhancing formulation.

In some embodiments, the aesthetic-enhancing formulation further includes one or more preservatives configured to prevent microbiological growth in the aesthetic-enhancing formulation.

Also disclosed herein is a packaged aesthetic-enhancing formulation including a container having a cap and an aesthetic-enhancing formulation sterilely sealed in the container. The aesthetic-enhancing formulation is configured for insertion into a dermal layer of skin by percutaneous punctures. The aesthetic-enhancing formulation includes one or more pigments, one or more active ingredients, and one or more excipients. The one-or-more excipients includes at least a vehicle configured to evenly distribute components throughout the aesthetic-enhancing formulation, the components including the one-or-more pigments, the one-or-more active ingredients, and any other excipients other than the vehicle.

In some embodiments, the container is a soft-sided container having a nipple disposed in an opening at a top of the container. The cap includes a nipple cap removably disposed over the nipple.

In some embodiments, the container is a soft-sided container. The cap includes a screwable nipple removably screwed over an opening at a top of the container and a nipple cap removably disposed over the nipple.

In some embodiments, the container is a rigidly sided container. The cap is crimped over an opening at a top of the container including a pierceable septum in a center of the cap.

Also disclosed herein is a method for enhancing aesthetics by simultaneously pigmenting a patient and administering one or more active ingredients to the patient. The method includes, in some embodiments, a device obtaining step of obtaining a pigmentation device having one or more needles. The method also includes a filling step of filling a reservoir of the pigmentation device with an aesthetic-enhancing formulation. The aesthetic-enhancing formulation includes one or more pigments, the one-or-more active ingredients, and one or more excipients. The one-or-more excipients includes at least a vehicle configured to evenly distribute components throughout the aesthetic-enhancing formulation, the components including the one-or-more pigments, the one-or-more active ingredients, and any other excipients other than the vehicle. The method also includes an inserting step of inserting the aesthetic-enhancing formulation into a dermal layer of a portion of skin by a plurality of percutaneous punctures with the one-or-more needles of the pigmentation device.

In some embodiments, the method further includes a preparing step of preparing the aesthetic-enhancing formulation. The preparing step includes a pigment obtaining step of obtaining the one-or-more pigments, a slurrying step of slurrying the one-or-more pigments in the vehicle to form a slurry, and an admixing step of admixing the one-or-more active ingredients to form the aesthetic-enhancing formulation from the slurry.

In some embodiments, the method further includes a choosing step of choosing the one-or-more pigments to match a natural color of human hair. The portion of skin into which the aesthetic-enhancing formulation is inserted during the inserting step is a scalp or an eyebrow region of the patient.

In some embodiments, the method further includes a choosing step of choosing the one-or-more pigments to match a natural color of human skin. The portion of skin into which the aesthetic-enhancing formulation is inserted during the inserting step is a scar, a stretch mark, a vitiligo patch, or a breast of the patient in need of nipple or areola restoration.

In some embodiments, the vehicle is water, hamamelis water, ethanol, isopropyl alcohol, propylene glycol, glycerol, or a combination thereof.

In some embodiments, the one-or-more active ingredients include a local anesthetic.

In some embodiments, the one-or-more active ingredients include stem cells, platelet-rich plasma, extracellular vesicles, a peptide hormone, a growth factor, collagen, nutrients, or a combination thereof.

These and other features of the concepts provided herein will become more apparent to those of skill in the art in view of the accompanying drawings and following description, which describe particular embodiments of such concepts in greater detail.

### DRAWINGS

FIG. 1 illustrates insertion of an aesthetic-enhancing formulation into a dermal layer of a portion of skin by a percutaneous puncture with a needle of a pigmentation device in accordance with some embodiments.

FIG. 2 provides images corresponding to descriptions of hair loss or scarring in men under the classification system set forth in Table 1.

FIG. 3 provides images corresponding to descriptions of hair loss or scarring in women under the classification system set forth in Table 2.

FIG. 4 provides images corresponding to descriptions of scarring in men or women under the classification system set forth in Table 3.

FIG. 5 provides images corresponding to descriptions of cosmetic defects in areolas or nipples under the classification system set forth in Table 4.

FIG. 6 illustrates a first packaged aesthetic-enhancing formulation in accordance with some embodiments.

FIG. 7 illustrates a second packaged aesthetic-enhancing formulation in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

Concentrations are generally expressed herein in terms of percent concentration. For example, a concentration for a component of the aesthetic-enhancing formulation can be expressed herein by percentage of the component solvated, suspended, or otherwise distributed throughout the aesthetic-enhancing formulation, wherein the component is measured by either volume or weight, and wherein the solution, suspension, or the like is also measured by either volume or weight. For example, a suspension of 5% (w/w) pigment is 5 g pigment in 100 g of the suspension:

$$\frac{5 g p i g m e n t}{100 g s u s p e n s i o n} \times 100 = 5 \% \left(w / w\right) p i g m e n t$$

Weight-percent concentration is favored over volume-percent concentration, weight/volume-percent concentration, or volume/weight-percent concentration by virtue of weight being temperature independent. However, it should be understood any weight-percent concentration disclosed herein can be alternatively interpreted as any percent concentration of volume-percent concentration, weight/volume-percent concentration, or volume/weight-percent concentration, thereby extending the disclosure without burdening the disclosure. It should also be understood such an interpretation does not extend to the claims; that is, the claimed percent concentration of any component of the aesthetic-enhancing formulation should be construed as claimed.

Unless defined otherwise, all technical and scientific terms used herein have the same meaning as commonly understood by those of ordinary skill in the art.

As set forth above, micropigmentation is increasingly being used for aesthetic enhancement of the body, particularly to conceal scars, stretch marks, or vitiligo patches, create or restore nipples or areolas, create or enhance hair follicles on scalps, eyebrow regions, or alopecia spots, or the like. While micropigmentation is effective for the foregoing types of aesthetic enhancement, micropigmentation consists of inserting pigments into the skin with a microneedler without any therapeutic treatment of the underlying conditions leading people to seek the foregoing types of aesthetic enhancement in the first place. Aesthetic enhancement including therapy is needed to treat such underlying conditions more effectively.

Disclosed herein are several embodiments of aesthetic-enhancing formulations or methods that meet at least the foregoing need, which enable patients to undergo a single procedure to obtain the results required of two different procedures. The aesthetic aesthetic-enhancing formulations are described first followed by packaged aesthetic-enhancing formulations and methods of the aesthetic-enhancing formulations.

### Aesthetic-Enhancing Formulations

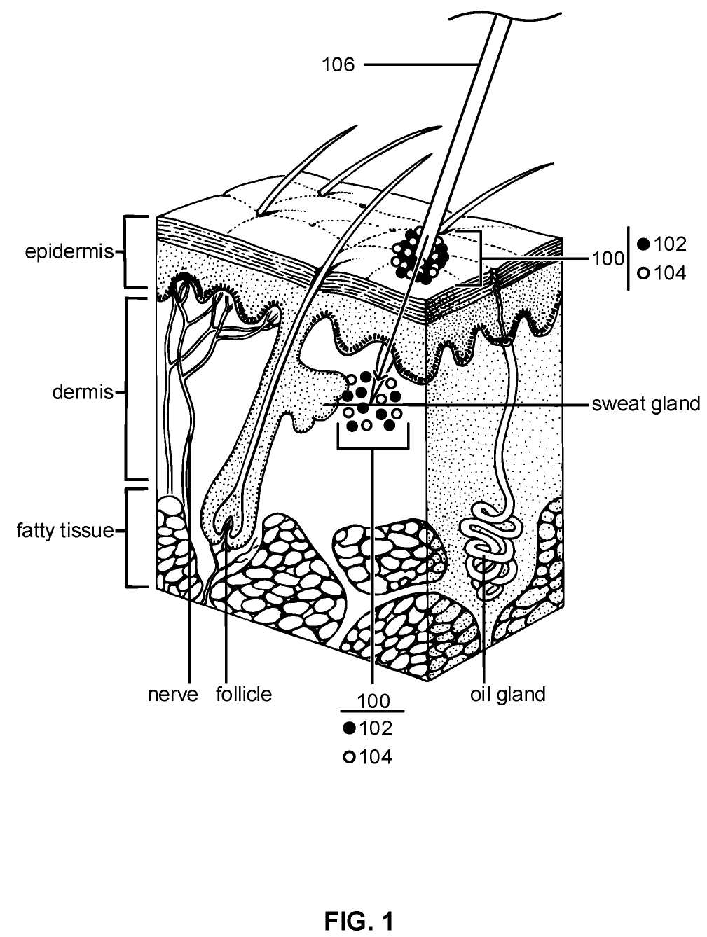

As shown in FIG. 1, an aesthetic-enhancing formulation 100 is configured for insertion into a dermal layer of a portion of skin by percutaneous punctures with at least a needle 106 such as needle of a pigmentation device. The aesthetic-enhancing formulation 100 includes one or more pigments 102, one or more active ingredients 104, and one or more excipients, each of which is set forth in more detail below. So as to not burden the disclosure, reference numbers are used only when directly referencing the figures.

Each pigment of the one-or-more pigments is preferably a plant-based pigment but is not limited thereto. The one-or-more pigments can be chosen to match a natural color of human hair in order to create or enhance hair follicles on scalps, eyebrow regions, or alopecia spots, or the like. The one-or-more pigments can be chosen to match a natural color of human skin in order to conceal scars, stretch marks, or vitiligo patches, create or restore nipples or areolas, or the like.

With respect to the one-or-more pigments in the aesthetic-enhancing formulation, the aesthetic-enhancing formulation can include at least about 1% (w/w), 5% (w/w), 10% (w/w), 15% (w/w), 20% (w/w), 25% (w/w), 30% (w/w), 35% (w/w), 40% (w/w), 45% (w/w), 50% (w/w), 55% (w/w), 60% (w/w), 65% (w/w), 70% (w/w), 75% (w/w), or an intervening concentration thereof, of each pigment of the one-or-more pigments or a combination of pigments in the aesthetic-enhancing formulation. Alternatively, the aesthetic-enhancing formulation can include no more than about 75% (w/w), 70% (w/w), 65% (w/w), 60% (w/w), 55% (w/w), 50% (w/w), 45% (w/w), 40% (w/w), 35% (w/w), 30% (w/w), 25% (w/w), 20% (w/w), 15% (w/w), 10% (w/w), 5% (w/w), or 1% (w/w), or an intervening concentration thereof, of each pigment of the one-or-more pigments or a combination of pigments in the aesthetic-enhancing formulation.

In view of the foregoing pigment concentrations, the aesthetic-enhancing formulation can include a pigment concentration range of at least about 1% (w/w) and no more than about 75% (w/w), or an intervening concentration thereof, for any given pigment of the one-or-more pigments or a combination of pigments in the aesthetic-enhancing formulation.

Each active ingredient of the one-or-more active ingredients is independently selected from biotechnological agents such as stem cells, platelet-rich plasma, extracellular vesicles (e.g., exosomes), peptide hormones (e.g., human growth hormone), growth factors (e.g., growth factor proteins), or collagen; small molecules such as small-molecule growth factors (e.g., steroid hormones such as finasteride), local anesthetics (e.g., benzocaine, lidocaine, tetracaine, bupivacaine, etc.), antibiotics (e.g., neomycin, polymyxin B, bacitracin, gramicidin, etc.), or analgesics (e.g., pramoxine); various nutrients such as vitamins (e.g., vitamin A, any one or more vitamins of the B vitamins such as biotin, vitamin C, vitamin D, vitamin E, etc.), “minerals” (e.g., iron such as iron(II) salts or iron(II) oxide, zinc such as zinc oxide, zinc acetate, or zinc gluconate, etc.), peptides or amino acids (e.g., cysteine, methionine, lysine, glycine, arginine, tyrosine, glutamine, proline, etc.), or essential fatty acids (e.g., omega-3 fatty acids); or a combination thereof.

With respect to the one-or-more active ingredients in the aesthetic-enhancing formulation, the aesthetic-enhancing formulation can include at least about 1% (w/w), 5% (w/w), 10% (w/w), 15% (w/w), 20% (w/w), 25% (w/w), 30% (w/w), 35% (w/w), 40% (w/w), 45% (w/w), 50% (w/w), 55% (w/w), 60% (w/w), or 65% (w/w), 70% (w/w), or 75% (w/w) or an intervening concentration thereof, of each active ingredient of the one-or-more active ingredients or a combination of active ingredients in the aesthetic-enhancing formulation. Alternatively, the aesthetic-enhancing formulation can include no more than about 75% (w/w), 70% (w/w), 65% (w/w), 60% (w/w), 55% (w/w), 50% (w/w), 45% (w/w), 40% (w/w), 35% (w/w), 30% (w/w), 25% (w/w), 20% (w/w), 15% (w/w), 10% (w/w), 5% (w/w), or 1% (w/w), or an intervening concentration thereof, of each active ingredient of the one-or-more active ingredients or a combination of active ingredients in the aesthetic-enhancing formulation.

In view of the foregoing active-ingredient concentrations, the aesthetic-enhancing formulation can include an active-ingredient concentration range of at least about 1% (w/w) and no more than about 75% (w/w), or an intervening concentration thereof, for any given active ingredient of the one-or-more active ingredients or a combination of active ingredients in the aesthetic-enhancing formulation.

Each excipient of the one-or-more excipients is independently selected from pharmaceutical excipients and nutraceutical excipients. However, the one-or-more excipients includes at least a vehicle (e.g., water, hamamelis water, ethanol, isopropyl alcohol, propylene glycol, glycerol, or a combination thereof) configured to evenly distribute components throughout the aesthetic-enhancing formulation, the components including the one-or-more pigments, the one-or-more active ingredients, and any other excipients other than the vehicle.

With respect to the one-or-more excipients in the aesthetic-enhancing formulation, the aesthetic-enhancing formulation can include at least about 1% (w/w), 5% (w/w), 10% (w/w), 15% (w/w), 20% (w/w), 25% (w/w), 30% (w/w), 35% (w/w), 40% (w/w), 45% (w/w), 50% (w/w), 55% (w/w), 60% (w/w), or 65% (w/w), 70% (w/w), or 75% (w/w) or an intervening concentration thereof, of each excipient of the one-or-more excipients or a combination of excipients in the aesthetic-enhancing formulation. Alternatively, the aesthetic-enhancing formulation can include no more than about 75% (w/w), 70% (w/w), 65% (w/w), 60% (w/w), 55% (w/w), 50% (w/w), 45% (w/w), 40% (w/w), 35% (w/w), 30% (w/w), 25% (w/w), 20% (w/w), 15% (w/w), 10% (w/w), 5% (w/w), or 1% (w/w), or an intervening concentration thereof, of each excipient of the one-or-more excipients or a combination of excipients in the aesthetic-enhancing formulation.

In view of the foregoing excipient concentrations, the aesthetic-enhancing formulation can include an excipient concentration range of at least about 1% (w/w) and no more than about 75% (w/w), or an intervening concentration thereof, for any given excipient of the one-or-more excipients or a combination of excipients in the aesthetic-enhancing formulation.

The aesthetic-enhancing formulation can further include one or more stabilizers configured to prevent degradation of any component or combination of the components of the aesthetic-enhancing formulation. For example, the one-or-more stabilizers can include an antioxidant such as an oxygen scavenger or radical scavenger configured to prevent oxidative degradation of the aesthetic-enhancing formulation. In another example, the one-or-more stabilizers can include an emulsifier configured to prevent separation of the components of the aesthetic-enhancing formulation into layers of the aesthetic-enhancing formulation.

The aesthetic-enhancing formulation can further include one or more preservatives configured to prevent microbiological growth in the aesthetic-enhancing formulation. For example, the one-or-more preservatives can include benzyl alcohol, salicylic acid, sorbic acid, a paraben, or a combination thereof.

### Packaged Aesthetic-Enhancing Formulations

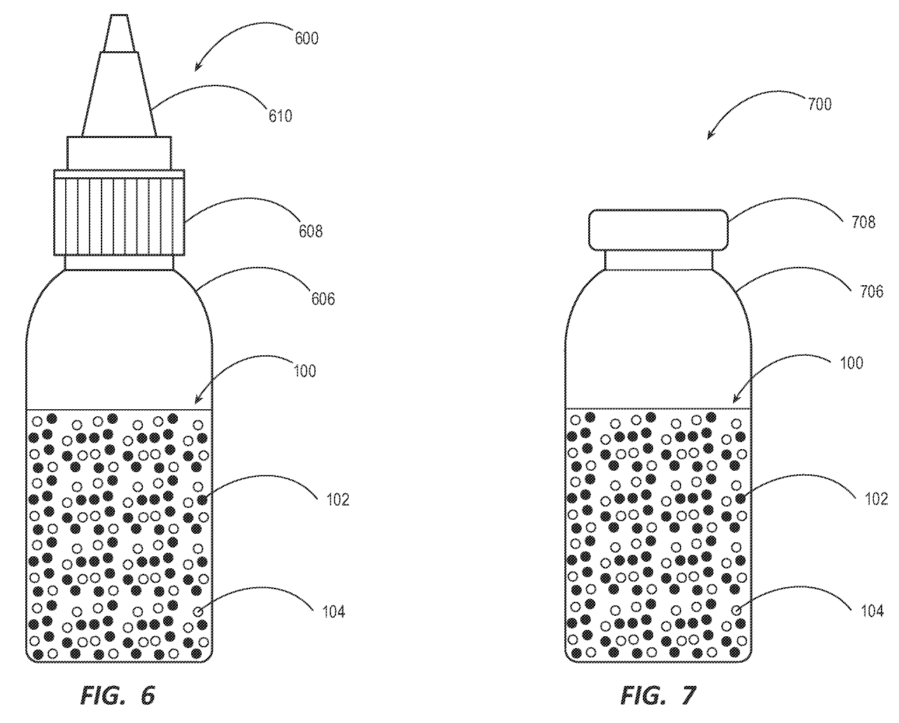

FIGS. 6 and 7 respectively illustrate packaged aesthetic-enhancing formulations 600 and 700 in accordance with some embodiments.

A packaged aesthetic-enhancing formulation such as the packaged aesthetic-enhancing formulation 600 or 700 includes a container 606 or 706 having a cap 608 or 708 and an aesthetic-enhancing formulation (e.g., the aesthetic-enhancing formulation 100) set forth above sterilely sealed in the container 606 or 706.

The container 606 can be a soft-sided, squeezable container (e.g., a plastic bottle). The container 606 can include a nipple (e.g., a plastic nipple) disposed in or otherwise integrated into an opening at a top of the container 606. A cap for such a container includes a nipple cap removably disposed over the nipple. Alternatively, the container 606 can include a threaded opening at a top of the container 606 as shown in FIG. 6. The cap 608 for such a container includes a threaded nipple removably screwed over the opening of the container 606, as well as a nipple cap 610 removably disposed over the nipple.

The container 706 can be a rigidly sided container (e.g., a glass bottle). The container 706 can include a flanged opening at a top of the container 706. The cap 708 for such a container includes a crimped cap crimped over the opening of the container 706, the cap 708 including a pierceable septum in a center of the cap 708.

### Methods

A method for enhancing aesthetics includes simultaneously pigmenting a patient and administering one or more active ingredients to the patient by way of an aesthetic-enhancing formulation set forth above.

The method can include a device obtaining step of obtaining a pigmentation device having one or more needles.

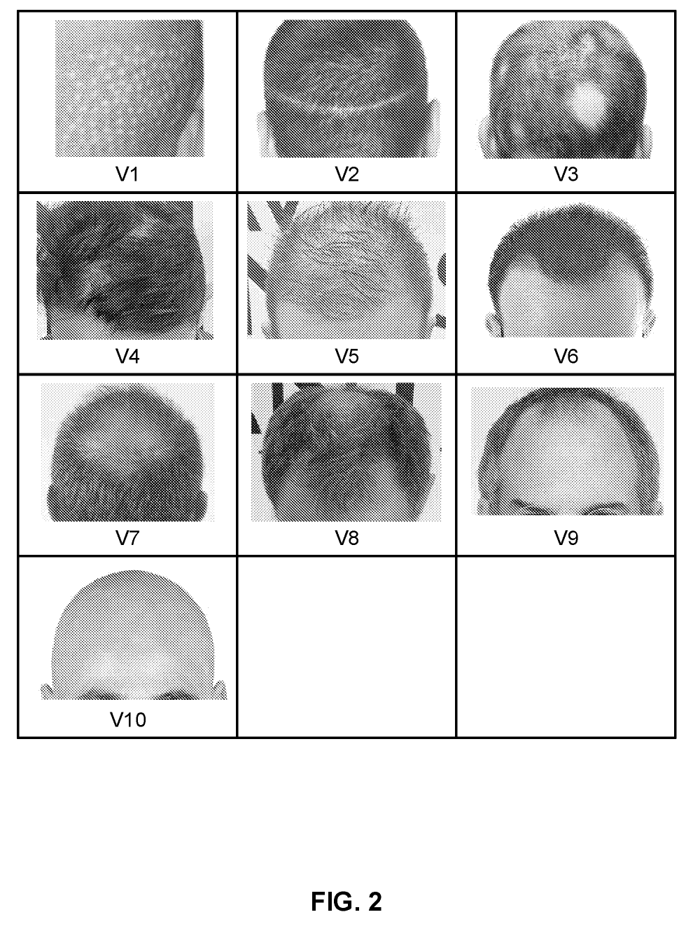

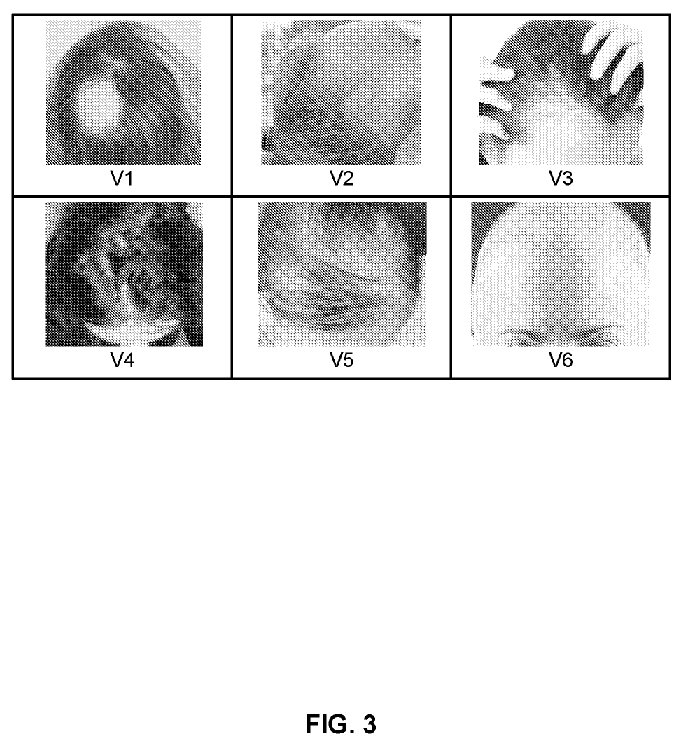

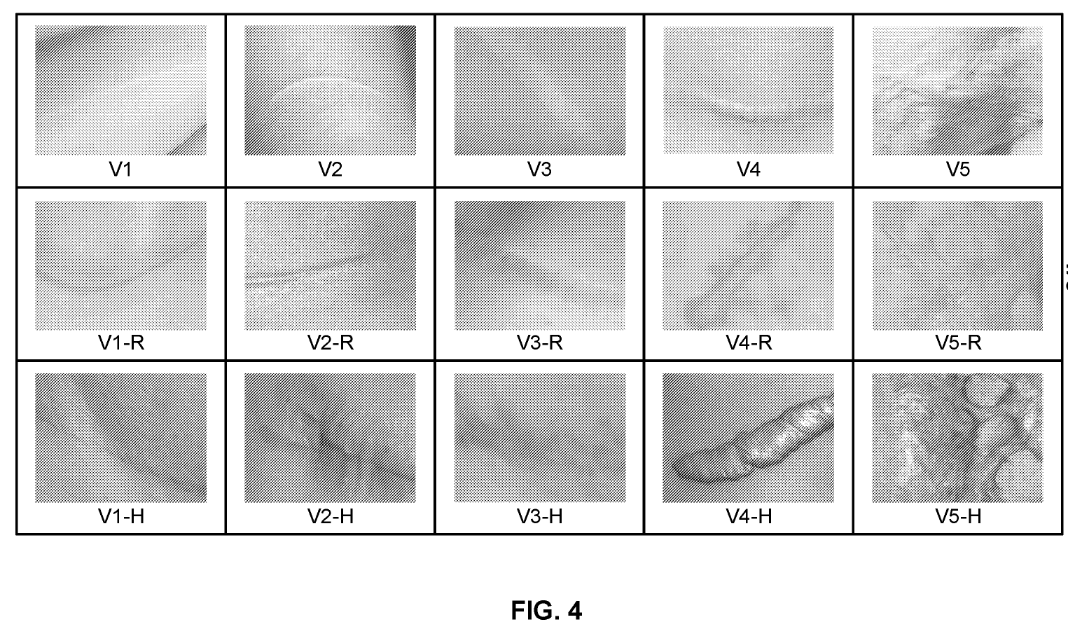

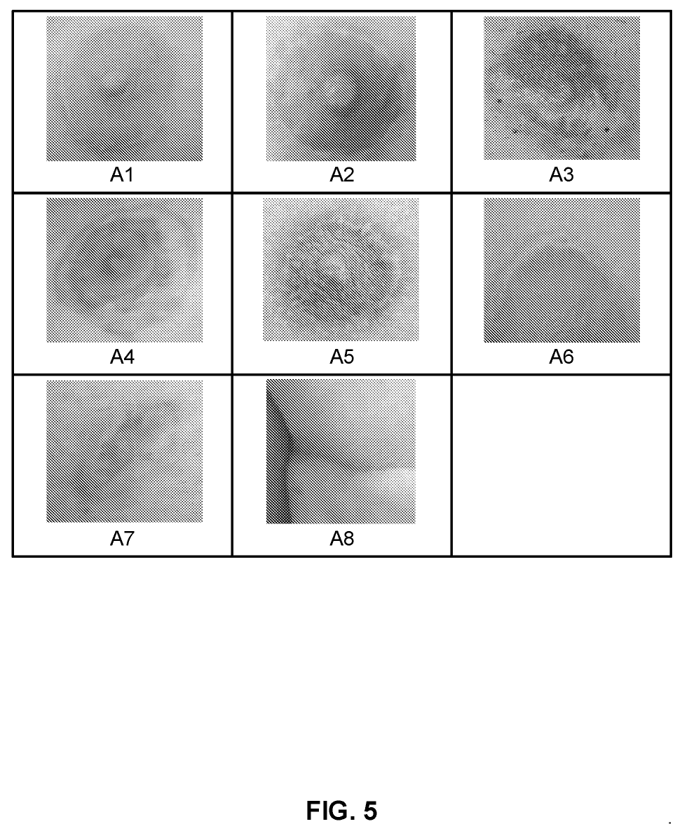

The method can include a classification step of classifying hair loss, scarring or the like in accordance with the classification systems for men, women, scarring, and cosmetic defects in areolas or nipples set forth in Tables 1-4 and corresponding FIGS. 2-5. The classification step is useful to a practitioner for adhering to best practices in treating hair loss, scarring, cosmetic defects in areolas or nipples, or the like in accordance with precedent. The classification step is also useful to a patient for understating one or more potential outcomes in accordance with precedent when the one-or-more potential outcomes are communicated to the patient by the practitioner. The “Type” in each of the tables is exemplary of potential designations for each category, but it should be appreciated that other designations or abbreviations are equally possible.

Table 1

Classification System for Men

| | |
|---|---|
| Type | Description |
| VF1 | Follicular unit extraction (“FUE”) scarring |
| | Small white dots of hair missing due to a previous FUE surgery |
| VF2 | Follicular unit transplant (“FUT”) scarring and miscellaneous scars |
| | Scarring caused by FUT surgery, other incisions, or injuries |
| VF3 | Alopecia |
| | One or more bald patches within hairline |
| VF4 | Thinning |
| | Hairline intact but hair thinning throughout top of head; no previous transplant surgeries |
| VF5 | Thinning after hair transplant |
| | Hairline might be weak but intact; hair thinning throughout top of head |
| VF6 | Receding hairline |
| | Hairline receding about lateral temples of head |
| VF7 | Crown |
| | Hair thinning or lost in crown region of head |
| VF8 | Receding and crown |
| | Hairline receding in front of head with hair thinning or lost in crown region of head; possible hair thinning that merges regions of the head |
| VF9 | Horseshoe effect |
| | Severe hair loss on top of head causing surrounding hair to appear like a horseshoe |
| VF10 | Bald |
| | Complete hair loss |

Table 2

Classification System for Women

| | |
|---|---|
| Type | Description |
| VF1 | Alopecia (medical) |
| | One or more bald patches within hairline often caused by auto-immune disorder, stress, hormones, or other medical conditions |
| VF2 | Tension alopecia |
| | One or more bald patches caused by tension often caused by certain types of hairstyle that involve pulling, extensions, etc. |
| VF3 | Receding hairline |
| | Hairline receding about lateral temples of head; common for women to only notice fine baby hairs about lateral temples when experiencing this type of hair loss |
| VF4 | Top thinning |
| | Hair thinning specifically around or near center head; often in addition to some hair thinning near crown region of head |
| VF5 | Global thinning |
| | Hair loss over entire head with visible scalp showing regardless of hairstyle |
| VF6 | Moderate to severe balding |
| | Severe hair loss over entire head; most patients shave their head as a result |

Table 3

Classification System for Scars

| | |
|---|---|
| Type | Description |
| V1 | Ideal scar with hypopigmentation |
| | Scar is thin and flat with no difference in texture compared to surrounding skin; overall hypopigmentation |
| V1-R | Ideal scar with red tones |
| | Ideal scar as above but red or with red borders |
| V1-H | Ideal scar with hyperpigmentation |
| | Ideal scar as above but has darker brown tones. |
| V2 | Mild-textured scar |
| | Scar is generally thin; appears like a crease, mild bumps, or slightly elevated; overall hypopigmentation |
| V2-R | Mild-textured scar with red tones |
| | Mild-textured scar as above but red or with red borders |
| V2-H | Mild-textured scar with hyperpigmentation |
| | Mild-textured scar as above but has darker brown tones |
| V3 | Widened scar (hypertrophic scar) |
| | Scar is thinner or thicker than surrounding skin with a visible difference in texture; overall hypopigmentation |
| V3-R | Widened scar with red tones |
| | Widened scar as above but is red in tone or has purple undertones |
| V3-H | Widened scar with hyperpigmentation |
| | Widened scar as above but has darker brown tones |
| V4 | Elevated scar |
| | Scar is firm with moderate to severe elevation that might distort scarring; overall hypopigmentation with occasional mild pink tones |
| V4-R | Elevated scar with red tones |
| | Elevated scar as above but is red or purple in tone |
| V4-H | Elevated scar with hyperpigmentation |
| | Elevated scar as above but has darker brown tones |
| V5 | Post-trauma scar |
| | Scar is often distorted in shape and surrounding skin is severely damaged with extreme differences in texture; often caused by wound openings, infections, burns, etc.; overall hypopigmentation |
| V5-R | Post-trauma scar with red tones |
| | Post-trauma scar as above but with is red or purple in tone |
| V5-H | Post-trauma scar with hyperpigmentation |
| | Post-trauma scar but has darker brown tones |

Table 4

Classification System for Defects in Areolas or Nipples

| | |
|---|---|
| Type | Description |
| A1 | Periareolar defect |
| | One or more bald patches within hairline often caused by auto-immune disorder, stress, hormones, or other medical conditions |
| A2 | Donut scar |
| | Scar around entire circumference of areola |
| A3 | Loss of pigment |
| | Areola appears patchy or misshapen due to loss of pigment within areola |
| A4 | Widened scar |
| | Hypertrophic scarring extending outside areola |
| A5 | Hyperpigmented scar |
| | Scar appears darker outside areola |
| A6 | Keloid scar |
| | Scar has moderate to severe elevation; appears pink or has darker tones |
| A7 | Post-wound breakdown or necrosis |
| | Areola appears misshapen, texture is uneven, and pigment is missing; depends upon degree of wound or loss of nipple |
| A8 | Mastectomy |
| | Areola and nipple are missing or surgically reconstructed |

The method can include a preparing step of preparing the aesthetic-enhancing formulation if a packaged aesthetic-enhancing formulation is not used. The preparing step includes a pigment obtaining step of obtaining one or more pigments, a slurrying step of slurrying the one-or-more pigments in a vehicle to form a slurry, and an admixing step of admixing one or more active ingredients to form the aesthetic-enhancing formulation from the slurry.

The pigment obtaining step includes a choosing step of choosing the one-or-more pigments to match a natural color of human hair or human skin. If a portion of skin into which the aesthetic-enhancing formulation is to be inserted is a scalp or an eyebrow region of the patient, the choosing step includes choosing the natural color of the hair of the patient about the portion of skin being treated. If a portion of skin into which the aesthetic-enhancing formulation is to be inserted during is a scar, a stretch mark, a vitiligo patch, or a breast of the patient in need of nipple or areola restoration, the choosing step includes choosing the natural color of the skin of the patient about the portion of skin being treated.

The method can include a filling step of filling a reservoir or tube of the pigmentation device with the aesthetic-enhancing formulation. Filling the reservoir or tube can include operating the pigmentation device while the one-or-more needles are disposed in the aesthetic-enhancing formulation.

The method includes an inserting step of inserting the aesthetic-enhancing formulation into a dermal layer of the portion of skin being treated by a plurality of percutaneous punctures with the one-or-more needles of the pigmentation device.

Because an aesthetic-enhancing session can take several hours (e.g., up to 6 about hours) any one or more steps of the method can be performed a number of times per session to effectuate a desired aesthetic. In addition, any one or more steps of the method can be performed over a number of aesthetic-enhancing sessions (e.g., up to about 5 sessions) to effectuate a desired aesthetic. It should be understood any step of the method performed need not be as previously performed if that step is performed a number of times over a session or across sessions. For example, the choosing step of choosing the one-or-more pigments can include matching a natural color of human skin in a first session and matching a natural color of human hair in a second session. In another example, the inserting step of inserting the aesthetic-enhancing formulation into the dermal layer of the skin can include inserting an alternative aesthetic-enhancing formulation lacking one or more pigments into the dermal layer of the skin. Such an alternative aesthetic-enhancing formulation is useful when a desired level of pigmentation has already been achieved in a session or across sessions.

### EXAMPLES

### Example 1: Pigmentation with Local Anesthetic

An aesthetic-enhancing formulation including pigment and lidocaine was prepared as set forth above and tested on pig skin. It was determined the inclusion of lidocaine did not affect pigment tone or consistency after treatment of the pig skin. Subsequently, a small test patch (e.g., a small dot) was tested on human skin (e.g., a human wrist) to test the efficacy of the lidocaine in the aesthetic-enhancing formulation. Within minutes the skin was numbed, thereby providing a qualitative determination of the efficacy. In addition, it was determined the inclusion of lidocaine did not affect pigment tone or consistency of the human skin.

### Example 2: Pigmentation with Biotechnological Agents

A first aesthetic-enhancing formulation including only pigment and a second aesthetic-enhancing formulation including pigment and platelet-rich plasma were prepared as set forth above and tested on two different portions of pig skin. It was determined the inclusion of platelet-rich plasma did not affect pigment tone or consistency after treatment of the pig skin as the two different portions of the pig skin looked identical after treatment of the pig skin.

A third aesthetic-enhancing formulation including pigment and exosomes was prepared as set forth above. The first aesthetic-enhancing formulation including only the pigment and the third aesthetic-enhancing formulation were tested on two different portions of pig skin. It was determined the inclusion of the exosomes did not affect pigment tone or consistency after treatment of the pig skin as the two different portions of the pig skin looked identical after treatment of the pig skin.

Subsequently, a small test patch was tested on human skin (e.g., a human wrist). The test patch consisted of three dots, with each dot of the three dots uniquely corresponding to an aesthetic-enhancing formulation of the first, second, and third aesthetic-enhancing formulations. It was immediately determined the inclusion of the platelet-rich plasma in the second aesthetic-enhancing formulation and the exosomes in the third aesthetic-enhancing formulation did not affect pigment tone or consistency of the human skin. The test patch was observed again at 6 weeks, 3 months, and 6 months thereafter. It was determined the inclusion of the platelet-rich plasma in the second aesthetic-enhancing formulation and the exosomes in the third aesthetic-enhancing formulation did not affect pigment tone or consistency of the human skin even after 6 months.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

1. An aesthetic-enhancing formulation, comprising:
one or more pigments;
one or more active ingredients; and
one or more excipients including at least a vehicle configured to evenly distribute components of the aesthetic-enhancing formulation including the one-or-more pigments, the one-or-more active ingredients, and any other excipients other than the vehicle throughout the aesthetic-enhancing formulation, the aesthetic-enhancing formulation configured for insertion into a dermal layer of skin by percutaneous punctures.

2. The aesthetic-enhancing formulation of claim 1, wherein each pigment of the one-or-more pigments is a plant-based pigment.

3. The aesthetic-enhancing formulation of claim 1 , wherein the one-or-more pigments are chosen to match a natural color of human hair.

4. The aesthetic-enhancing formulation of claim 1 , wherein the one-or-more pigments are chosen to match a natural color of human skin.

5. The aesthetic-enhancing formulation of claim 1 , wherein the one-or-more active ingredients include a local anesthetic.

6. The aesthetic-enhancing formulation of claim 1 , wherein the one-or-more active ingredients include stem cells, platelet-rich plasma, extracellular vesicles, a peptide hormone, a growth factor, collagen, nutrients, or a combination thereof.

7. The aesthetic-enhancing formulation of claim 1 , wherein the vehicle is water, hamamelis water, ethanol, isopropyl alcohol, propylene glycol, glycerol, or a combination thereof.

8. The aesthetic-enhancing formulation of claim 1 , further comprising one or more stabilizers configured to prevent degradation of any component or combination of the components of the aesthetic-enhancing formulation.

9. The aesthetic-enhancing formulation of claim 8, wherein the one-or-more stabilizers include an antioxidant to prevent oxidative degradation of the aesthetic-enhancing formulation.

10. The aesthetic-enhancing formulation of claim 1 , wherein the one-or-more stabilizers include an emulsifier to prevent separation of the components of the aesthetic-enhancing formulation into layers of the aesthetic-enhancing formulation.

11. The aesthetic-enhancing formulation of claim 1 , further comprising one or more preservatives configured to prevent microbiological growth in the aesthetic-enhancing formulation.

12. A packaged aesthetic-enhancing formulation, comprising:
a container having a cap; and
an aesthetic-enhancing formulation sterilely sealed in the container, the aesthetic-enhancing formulation including:
one or more pigments;
one or more active ingredients; and
one or more excipients including at least a vehicle configured to evenly distribute components of the aesthetic-enhancing formulation including the one-or-more pigments, the one-or-more active ingredients, and any other excipients other than the vehicle throughout the aesthetic-enhancing formulation, the aesthetic-enhancing formulation configured for insertion into a dermal layer of skin by percutaneous punctures.

13. The packaged aesthetic-enhancing formulation of claim 12, wherein the container is a soft-sided container having a nipple disposed in an opening at a top of the container, the cap including a nipple cap removably disposed over the nipple.

14. The packaged aesthetic-enhancing formulation of claim 12, wherein the container is a soft-sided container, the cap including a screwable nipple removably screwed over an opening at a top of the container and a nipple cap removably disposed over the nipple.

15. The packaged aesthetic-enhancing formulation of claim 12, wherein the container is a rigidly sided container, the cap crimped over an opening at a top of the container including a pierceable septum in a center of the cap.

16. A method for enhancing aesthetics, comprising:
obtaining a pigmentation device having one or more needles;
filling a reservoir of the pigmentation device with an aesthetic-enhancing formulation, the aesthetic-enhancing formulation including:
one or more pigments;
one or more active ingredients; and
one or more excipients including at least a vehicle configured to evenly distribute components of the aesthetic-enhancing formulation including the one-or-more pigments, the one-or-more active ingredients, and any other excipients other than the vehicle throughout the aesthetic-enhancing formulation; and
inserting the aesthetic-enhancing formulation into a dermal layer of a portion of skin by a plurality of percutaneous punctures with the one-or-more needles of the pigmentation device, thereby simultaneously pigmenting a patient and administering the one-or-more active ingredients to the patient.

17. The method of claim 16, further comprising preparing the aesthetic-enhancing formulation, including:
obtaining the one-or-more pigments;
slurrying the one-or-more pigments in the vehicle to form a slurry; and
admixing the one-or-more active ingredients to form the aesthetic-enhancing formulation from the slurry.

18. The method of claim 16, further comprising choosing the one-or-more pigments to match a natural color of human hair, the portion of skin being a scalp or an eyebrow region of the patient.

19. The method of claim 16, further comprising choosing the one-or-more pigments to match a natural color of human skin, the portion of skin is a scar, a stretch mark, a vitiligo patch, or a breast of the patient in need of nipple or areola restoration.

20. The method of claim 16, wherein the vehicle is water, hamamelis water, ethanol, isopropyl alcohol, propylene glycol, glycerol, or a combination thereof.

21. The method of claim 16, wherein the one-or-more active ingredients include a local anesthetic.

22. The method of claim 16, wherein the one-or-more active ingredients include stem cells, platelet-rich plasma, extracellular vesicles, a peptide hormone, a growth factor, collagen, nutrients, or a combination thereof.
